(19) United States
(12) Patent Application Publication
(10) Pub. No.: US 2023/0142738 A1
(43) Pub. Date: May 11, 2023

(54) ELECTROCHROMIC MATERIAL AND PREPARATION METHOD THEREOF, ELECTROCHROMIC DEVICE

(71) Applicant: Micron Optoelectronics Co., Ltd., Shenzhen (CN)

(72) Inventors: Wei SU, Shenzhen (CN); Guoliang ZHANG, Shenzhen (CN); Shourong HU, Shenzhen (CN)

(73) Assignee: Micron Optoelectronics Co., Ltd., Shenzhen (CN)

(21) Appl. No.: 17/980,007

(22) Filed: Nov. 3, 2022

(30) Foreign Application Priority Data
Nov. 10, 2021 (CN) 202111336028.9

(51) Int. Cl. C09K 9/02

(52) U.S. Cl. CPC C09K 9/02; C09K 2211/187

(57) ABSTRACT

Disclosed is an electrochromic material. The electrochromic material includes: a low eutectic solvent, and a color-changing material which includes a hydrogen bond donor material and a hydrogen bond acceptor material. The electrochromic material also includes an aerogel.

### CROSS-REFERENCE TO RELATED APPLICATIONS

This application claims priority to Chinese Patent Application No. 202111336028.9, filed on Nov. 10, 2021, the entire contents of which are incorporated herein by reference.

### TECHNICAL FIELD

The present application relates to the technical field of optoelectronic materials, in particular to an electrochromic material and a preparation method thereof, an electrochromic device.

### BACKGROUND

Electrochromic refers to the phenomenon that a stable and reversible redox reaction occurs under the action of electric current or electric field, and a reversible change of color or transparency occurs on the appearance. Devices prepared from electrochromic materials have the characteristics of user-controllable color change, low power consumption, low operating voltage, continuously adjustable optical performance, green and intelligent, etc. They have been widely used in the fields of electrochromic smart windows, automatic anti-glare rearview mirrors of the vehicle, color-changing sunglasses, information displays, etc.

Electrochromic devices are electrochemical devices, which include at least two electrodes and an electrolyte layer. Typically, the electrochromic material can be dissolved in the electrolyte or coated on one or two electrodes. Electrochemical displays have attracted a lot of attention due to their simplicity in device configuration and fabrication, low-voltage operation and low power consumption, and many studies have been done in recent years, but these studies are based on ionic liquid electrochromic devices. Compared with ionic liquids, low eutectic solvents have excellent characteristics: low price, low toxicity, green environment, stable physical and chemical properties, good electrical conductivity, and wide electrochemical window.

### SUMMARY

The present application proposes an electrochromic material, to improve the performance of electrochromic materials.

To achieve the above purpose, the present application provides an electrochromic material, including:

a low eutectic solvent; and

a color-changing material comprising a hydrogen bond donor material and a hydrogen bond acceptor material.

In an embodiment, the low eutectic solvent includes a choline substance and a ligand, the choline substance includes one or more choline compounds, and the ligand comprises one or more ligands.

In an embodiment, the choline compound has a structural formula [N(CH₃)₃CH₂CH₂OH]+[X]—, X is a halogen or a hydroxyl group;

the ligand is a halogenated metal salt, a hydrated halogenated metal salt or a compound with a structural formula R1-R2;

R1 is an alkyl group and R2 is an electron-donating group containing N, O, P, S or halogen atoms.

In an embodiment, the hydrogen bond acceptor material includes one or more of a viologen compound, a 9,10-anthraquinone compound.

In an embodiment, the viologen compound has a structural formula:

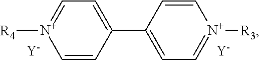

R3, R4 are alkyl or aromatic groups, Y⁻ is (PF₆)⁻, (AsF₆)⁻, (ClO₄)⁻, CH₃COO⁻, CH₃(C₆H₄)SO₃⁻, or halogen ions.

In an embodiment, the hydrogen bond acceptor material is one or more compounds of viologen, benzyl viologen, cyano-substituted phenyl viologen, amino-substituted phenyl viologen, acyloxy viologen, and carboxymethyl viologen.

In an embodiment, the hydrogen bond donor material is one or more compounds of ferrocene and derivatives of the ferrocene, phenothiazine and derivatives of the phenothiazine, triphenylamine and derivatives of the triphenylamine.

In an embodiment, the electrochromic material further includes an aerogel.

The present application also provides a method for preparing an electrochromic material, including:

(S1) drying a choline substance and a ligand, and mixing uniformly the choline substance and the ligand under heating conditions to obtain component I₁;

(S2) adding a color-changing material to the component I₁ and mixing uniformly to obtain component I₂; and

(S3) adding aerogel to I₂ and mixing uniformly to obtain component I₃.

The present application also provides an electrochromic device, including the electrochromic material as mentioned above.

Compared with the electrochromic materials of the prior art, the present application adopts the low eutectic solvent as electrolyte, and the electrochromic material with low eutectic solvent as electrolyte has the following advantages: low price, low toxicity, green environment, stable physical and chemical properties, good electrical conductivity, and wide electrochemical window.

### BRIEF DESCRIPTION OF THE DRAWINGS

In order to more clearly illustrate the technical solutions in the embodiments of the present application or in the prior art, the drawings in the description of the embodiments or prior art will be simply introduced as below. Obviously, the drawings in the following description are only some embodiments of the present application, and other structures can be obtained by those skilled in the art according to structures illustrated in these drawings without any creative work.

FIG. 1 is a structural schematic view of three states of viologen according to an embodiment of the present application.

FIG. 2 is a flowchart of a preparation method of the electrochromic material according to an embodiment of the present application.

The realization of the purpose, functional features and advantages of the present application will be further described with reference to the drawings, in conjunction with the embodiments.

### DETAILED DESCRIPTION OF THE EMBODIMENTS

The following is a further clear and complete description of the technical features in the technical solutions provided by the present application in conjunction with specific embodiments. Obviously, the described embodiments are only a part of the embodiments of the present application, and not all of them. Based on the embodiments in the present application, all other embodiments obtained by those skilled in the art without any creative labor fall within the scope of the present application.

It will be understood by those skilled in the art that, unless otherwise defined, all terms used herein (including technical terms and scientific terms) have the same meaning as generally understood by those skilled in the art to which the present application belongs. It also should be understood that terms such as those defined in a general dictionary are to be understood as having a meaning consistent with the meaning in the context of the prior art and, unless defined as herein, are not to be interpreted in an idealized or overly formal sense.

The terms “preferably”, “more preferably”, etc. in the present application refer to embodiments of the present application that may provide certain beneficial effects in certain circumstances. However, other embodiments may also be preferred in the same or other circumstances. Moreover, the expression of one or more preferred embodiments does not imply that other embodiments are not available, nor is it intended to exclude other embodiments from the scope of the present application.

In addition, the technical solutions among the various embodiments may be combined with each other, but only on the basis that they can be realized by those skilled in the art, and when the combination of technical solutions appears to be contradictory or unrealizable such combination of technical solutions shall be deemed not to exist and not to be within the scope of the present application.

The present application proposes an electrochromic material, the electrochromic material includes a low eutectic solvent and a color-changing material, the low eutectic solvent includes a choline substance and a ligand, the choline substance includes one or more choline compounds, the ligand includes one or more ligands, the low eutectic solvent includes a hydrogen bond donor material and a hydrogen bond acceptor material.

Specifically, the choline compound has the structural formula [N(CH₃)₃CH₂CH₂OH]⁺[X]⁻, where X is a halogen or a hydroxyl group, i.e. the choline compound may be any of choline, choline fluoride, choline chloride, choline bromide or choline iodide. The ligand is a halogenated metal salt, a hydrated halogenated metal salt or a compound with the structural formula R₁-R₂; wherein R₁ is an unsubstituted or substituted alkyl group and R₂ is an electron-donating group containing N, O, P, S atoms.

Furthermore, the halogenated salt is any one of sodium fluoride, sodium chloride, sodium bromide, sodium iodide, potassium fluoride, potassium chloride, potassium bromide, potassium iodide, magnesium fluoride, magnesium chloride, magnesium bromide, magnesium iodide, ferric chloride, ferric bromide, ferric iodide, calcium chloride, calcium bromide, calcium iodide, aluminum chloride, aluminum bromide, aluminum iodide, tin dichloride, tin tetrachloride, zinc chloride, zinc bromide, zinc iodide, and chromium trichloride.

The hydrated halogenated metal salt is any one of magnesium chloride hexahydrate, magnesium bromide hexahydrate, magnesium iodide octahydrate, potassium chloride dihydrate, potassium triiodide monohydrate, calcium chloride dihydrate, calcium chloride hexahydrate, aluminum chloride hexahydrate, ferric chloride hexahydrate, aluminum bromide hexahydrate, and tin chloride pentahydrate.

The R₁ is C1-C12 alkyl, such as methyl, ethyl, propyl, butyl, n-pentyl, iso-pentyl, neopentyl, octyl, allyl, and allylbutyl. The R₁ is any one of hydroxyl, primary amino, secondary amino, tertiary amino, nitro, cyano, carbonyl, alkoxy, amido, acyloxy, mercapto, thiocyano, isothiocyano, sulfonic acid, phosphate, and halogen group.

Further, the ligand is methanol, ethanol, propanol, n-butanol, isobutanol, n-amyl alcohol, isoamyl alcohol, neopentyl alcohol, ethylene glycol, butylene glycol, glycerol, trimethylolpropane, urea, glucose, xylitol, sorbitol, 2-ethyl-3-enol, ethylamine, ethylenediamine hexanediamine, diethylenetriamine, triethylenetetramine, ethylamino acetate, ethylenediaminetetraacetate, ethanethiol, propanethiol, allyl mercaptan, methyl sulfide, ethyl sulfide, diethyl dithiophosphate, phenyl phosphine, and any one of chlorinated alkanes, brominated alkanes, iodinated alkanes.

The low eutectic solvent includes one or more groups of solvents: choline/methanol, choline/ethanol, choline/n-butanol, choline/isobutanol, choline/n-amyl alcohol, choline/isopentyl alcohol, choline/neopentyl alcohol, choline/2-ethyl-3-enol, choline/ethylamine, choline/ethylenediamine, choline/hexanediamine, choline/diethylenetriamine, choline/triethylenetetramine, choline/ethylamino acetate, choline/ethylenediaminetetraacetate, choline/ethanethiol, choline/propanethiol, choline/allylthiol, choline/methylsulfide, choline/ethanethiol, choline/diethylphosphorodithioate, choline/phenylphosphine, choline/1,2-dichloroethane, choline/1,2-dibromoethane, choline/1,2-dibromo-3-chloropropane, choline/2-iodopropane, choline/iodobutane, choline/ethanol, choline chloride/n-butanol, choline chloride/isobutanol choline chloride/isobutanol, choline chloride/ethylenediamine, choline chloride/diethylenetriamine, choline chloride/triethylenetetramine, choline chloride/ethylaminoacetate, choline chloride/ethylenediaminetetraacetate, choline chloride/ethanol sulfur, choline chloride/alkenol sulfur, choline chloride/methylsulfide, choline chloride/ethyl sulfide, choline chloride/propyl sulfide, choline chloride/diethyl dithiophosphate, and choline chloride/phenylphosphine. It should be noted that in each group of solvents, the choline compound and the ligand are mixed uniformly in a ratio of a certain amount of substance, the ratio of the amount of substance of the choline compound to the amount of substance of the ligand depends on the coordinate number of the ligand, if the ligand is an n-dendate ligand, the ratio of the amount of substance of the choline compound to the amount of substance of the ligand is m:1, m:n=(1.01:1)-(1.5:1).

In the electrochromic material, the hydrogen bond acceptor material includes one or more compounds of the viologen compounds with the structural formula (I1).

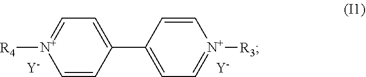

R₃, R₄ are alkyl or aromatic groups; Y⁻ is (PF₆)⁻, (AsF₆)⁻, (ClO₄)⁻, CH₃COO⁻, CH₃(C₆H₄)SO₃⁻, or halogen ions.

In more detail, R₃, R₄ are C1-C12 alkyl groups, such as: unsubstituted alkyl, dialkylamino, alkylamino, aminoalkyl, alkoxy, hydroxyalkyl, amido, amido-substituted alkyl, acyloxy, acyloxyalkyl, and carboxymethyl; or R3, R4 are C1-C16 aromatic groups, such as: benzyl, halogen-substituted phenyl, alkyl-substituted phenyl, amino-substituted phenyl, alkoxy-substituted phenyl, hydroxy-substituted phenyl, amido-substituted phenyl, acyloxy-substituted phenyl, and carboxymethyl-substituted phenyl.

For instance, the viologen compound can be viologen, benzyl viologen, cyanophenyl viologen, aminophenyl viologen, acyloxy viologen, and carboxymethyl viologen, according to the mentioned above.

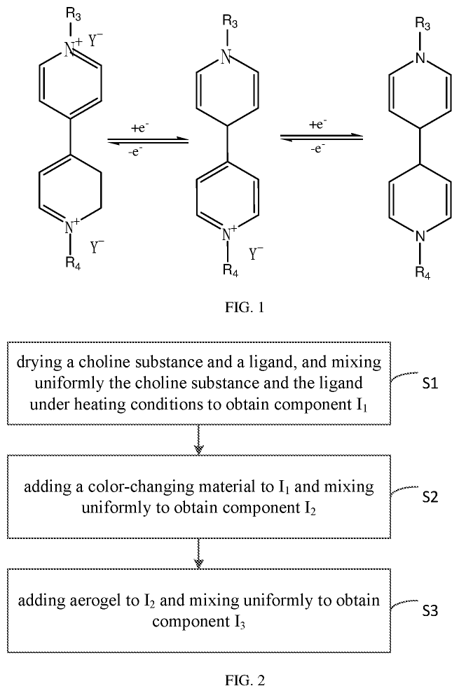

The viologen is a common hydrogen bond acceptor color-changing material, chemically named 1,1′-disubstituted-4,4′-bipyridine salt, exhibiting three reversible redox states; the viologen compound (I1)′ electrons under the action of light excitation or an applied electric field are transferred from anions (e.g., Cl⁻, Br⁻, I⁻, BF3⁻, PF6⁻) to the bipyridine ring to generate the viologen radical monovalent cation, the light charge on the bipyridine ring out of the domain makes the radical cation in the visible region has a high molar absorption coefficient, showing a very dark color; in the neutral state, the viologen compound has no absorption in the wavelength region of 400-800 nm, being colorless and transparent. Chemical modification of the N position in 4,4′-bipyridine with different functional groups can synthesize a series of viologen color-changing materials with different substituents for the purpose of multi-color display. FIG. 1 shows three states of viologen.

The hydrogen bond donor material is one or more compounds of ferrocene and derivatives of the ferrocene, phenothiazine and derivatives of the phenothiazine, triphenylamine and derivatives of the triphenylamine. The derivatives of the ferrocene, phenothiazine and triphenylamine can be made by substitution reactions of ferrocene, phenothiazine and triphenylamine with tertiary amine positive ion, nitro, trihalomethyl, cyano, sulfonic acid group, formyl, acyl, hydroxyl or carboxyl groups, respectively. The stronger the electron-absorbing ability of the substituted group, the more active the substituted ferrocene, substituted phenothiazine or substituted triphenylamine is in reacting with the hydrogen bond acceptor.

The ferrocene, phenothiazine and triphenylamine are all electrophilic, and their electrophilicity is even stronger after substitution by electron-absorbing groups, and the ferrocene, phenothiazine and triphenylamine are widely used in the field of electrochromic.

The color-changing materials consist of one or more hydrogen bond donor materials and one or more hydrogen bond acceptor materials, for example, the color-changing materials can be one or more of benzyl violet/hydroxyl ferrocene, cyano-substituted phenyl violet/hydroxyl ferrocene, amino-substituted phenyl violet/ferrocene, benzyl violet/phenothiazine, cyano-substituted phenyl violet/phenothiazine, amino-substituted phenyl violet/phenothiazine, benzyl violet/triphenylamine, and amino substituted phenyl viologen/triphenylamine.

The electrochromic materials also include aerogels. Aerogel is a form of solid matter with the smallest density in the world. There are many types of aerogels, including silicon-based, carbon-based, sulfur-based, metal oxide-based, metal-based, etc. In the preparation process of the electrochromic material, silicon-based aerogels, such as SiO₂-A380 aerogel powder, are generally used.

By adding aerogel into the electrochromic material, the electrochromic material is transformed from conventional liquid state to colloidal state, which avoids the electrolyte leakage problem of electrochromic devices due to external force, and further avoids many problems caused by electrolyte leakage.

As shown in FIG. 2, the present application also provides a preparation method of the electrochromic material, specifically, which includes:

S1: drying a choline substance and a ligand, and mixing uniformly the choline substance and the ligand under heating conditions to obtain component I₁;

S2: adding a color-changing material to I₁ and mixing uniformly to obtain component I₂;

S3: adding aerogel to I₂ and mixing uniformly to obtain component I₃.

In the above preparation process, firstly, the choline substance and the ligand are dried in a vacuum drying glove box, and then the choline substance and the ligand are heated at 50-60° C. and stirred evenly until a colorless and transparent liquid is obtained. Subsequently it is cooled to room temperature to obtain component I1, and no crystals are observed to precipitate at room temperature. Component I1 is sealed and kept in stock; the color-changing material is added to the prepared component I1, which is made into a solution containing 8 mmol/L-20 mmol/L hydrogen bond donor material (or hydrogen bond acceptor material), and then stirred and mixed uniformly to obtain component I2; aerogel is added to the prepared I2, and then stirred and mixed uniformly to obtain component I3 (that is, the electrochromic material). The mass of the aerogel is 5%-30% of the total mass of the color-changing material. Component I3 is sealed and packaged in a vacuum-drying glove box for light-proof treatment.

It should be noted that the low eutectic solvent may include one or more compounds of choline or choline halide, and one or more ligands; the color-changing material includes one or more hydrogen bond donors, and one or more hydrogen bond acceptors.

In the above preparation process, all steps need to be carried out in a vacuum-drying glove box because the components in the low eutectic solvent tend to react with water and air. In order to prevent the above conditions, the choline substances and ligands need to be dried before formulation, and then are mixed and formulated. At 50-60° C., the choline substance has better compatibility with the ligand, which is conducive to quickly mix the choline substance with the ligand uniformly.

The present application also provides an electrochromic device including the electrochromic material.

Since the electrochromic device adopts all of the above technical solutions, it has at least all of the beneficial effects brought about by the above technical solutions, which will not be repeated herein.

In order to further understand the present application, a kind of electrochromic material, a preparation method thereof, and electrochromic device provided by the present application will be described in detail below in combination with the embodiments, and the scope of the present application is not limited by the following embodiments.

### Embodiment 1

In a vacuum-drying glove box, the choline chloride and the ethylene glycol are mixed in the molar ratio of 2.5:1, heated and stirred at 60° C. until a colorless and transparent liquid is obtained, and then cooled to room temperature at which no crystals are observed to precipitate. 0.0107 g of cyanophenyl viologen and 0.0057 g of hydroxyferrocene are dissolved in 5 mL choline/ethylene glycol low eutectic solvent to obtain a solution containing 5 mmol/L cyanophenyl viologen and 5 mmol/L hydroxyferrocene. 1 mL of the above solution is added with 0.3 g of SiO₂-A380 with 10% mass fraction and stirred until the gel appears transparent to obtain a gel containing 5 mmol/L cyanophenyl viologen and 5 mmol/L hydroxyferrocene. At the same time, liquid amethyst-ionic liquid electrochromic material without SiO₂-A380 was also prepared as a control.

### Embodiment 2

In a vacuum-drying glove box, the choline chloride and ethylene glycol are mixed in the molar ratio of 1:2.5 and heated at 60° C. with constant stirring until a colorless and transparent liquid was obtained, and then cooled to room temperature. 0.0223 g of mono-substituted benzyl viologen and 0.0172 g of hydroxyferrocene are dissolved in 5 mL choline/ethylene glycol low eutectic solvent to obtain a solution containing 15 mmol/L monosubstituted benzyl violet extract and 15 mmol/L hydroxyferrocene. 1 mL of the above solution is added with 0.0158 g SiO₂-A380 with a 10% mass fraction and stirred until the gel appears transparent to obtain a gel containing 15 mmol/L benzyl viologen and 15 mmol/L hydroxyferrocene. At the same time, liquid mono-substituted benzyl viologen-choline/ethylene glycol electrochromic material without SiO₂-A380 was prepared as a control.

Control Group 1

Taking 5 mL 1-butyl-3methylimidazole bromide salt (ionic liquid) as electrolyte; 0.0223 g of mono-substituted benzyl viologen and 0.0172 g of hydroxyferrocene are dissolved in electrolyte to obtain a solution containing 15 mmol/L mono-substituted benzyl viologen and 15 mmol/L hydroxyferrocene. The electrochromic material containing 15 mmol/L benzyl viologen and 15 mmol/L hydroxyferrocene with ionic liquid as electrolyte is obtained from 1 mL the above solution.

TABLE 1
Results from embodiments and control group

| Embodiment | Embodiment 1 | | Embodiment 2 | | Control group 1 |
|---|---|---|---|---|---|
| state | liquid | solid | liquid | solid | liquid |
| color | colorless-light | | colorless- | | colorless-light |
| changing | green-dark green | | pink-fuchsia | | blue-dark blue |
| coloring | 193 | 233 | 48 | 65 | 38 |
| efficiency | | | | | |
| (cm²/C) | | | | | |
| cyclic test | 300 | 600 | 150 | 400 | 150 |
| transmittance | times | times | times | times | times |
| retention | | | | | |
| packaging | high | general | high | general | high |
| requirements of | | | | | |
| the color | | | | | |
| changing device | | | | | |

From Table 1, it can be concluded that compared to the electrochromic material using an ionic liquid as the electrolyte in the prior art, the electrochromic material made by using a low eutectic solvent as the electrolyte and the addition of aerogel has higher coloring efficiency, higher cyclic test life, lower packaging equipment for the color-changing device, and a synergistic effect.

The foregoing embodiments are merely illustrative and serve to explain some of the features of the method described herein. The appended claims are intended to claim the broadest conceivable scope and the embodiments presented herein are merely illustrative of selected implementations according to a combination of all possible embodiments. Accordingly, it is the intention of the applicant that the appended claims are not limited by the selection of examples that characterize the application. Some of the numerical ranges used in the claims also include sub-ranges within them, and variations within these ranges should also be construed, where possible, to be covered by the appended claims.

What is claimed is:

1. An electrochromic material, comprising:
a low eutectic solvent; and
a color-changing material comprising a hydrogen bond donor material and a hydrogen bond acceptor material.

2. The electrochromic material according to claim 1, wherein the low eutectic solvent comprises a choline substance and a ligand, the choline substance comprises one or more choline compounds, and the ligand comprises one or more ligands.

3. The electrochromic material according to claim 2, wherein the choline compound has a structural formula [N(CH₃)₃CH₂CH₂OH]⁺[X]⁻, wherein X is a halogen or a hydroxyl group;
the ligand is a halogenated metal salt, a hydrated halogenated metal salt or a compound with a structural formula R₁-R₂;
wherein R₁ is an alkyl group and R₂ is an electron-donating group containing N, O, P, S or halogen atoms.

4. The electrochromic material according to claim 1, wherein the hydrogen bond acceptor material comprises one or more of a viologen compound, and a 9,10-anthraquinone compound.

5. The electrochromic material according to claim 4, wherein the viologen compound has a structural formula: 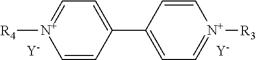
wherein R₃, R₄ are alkyl or aromatic groups;
Y is (PF₆)⁻, (AsF₆)⁻, (ClO₄)⁻, CH₃COO⁻, CH₃(C₆H₄)SO₃⁻, or halogen ions.

6. The electrochromic material according to claim 5, wherein the hydrogen bond acceptor material is one or more compounds of viologen, benzyl viologen, cyano-substituted phenyl viologen, amino-substituted phenyl viologen, acyloxy viologen, and carboxymethyl viologen.

7. The electrochromic material according to claim 1, wherein the hydrogen bond donor material is one or more compounds of ferrocene and derivatives of the ferrocene, phenothiazine and derivatives of the phenothiazine, triphenylamine and derivatives of the triphenylamine.

8. The electrochromic material according to claim 1, further comprising an aerogel.

9. A method for preparing an electrochromic material, comprising:
drying a choline substance and a ligand, and mixing the choline substance and the ligand uniformly under heating conditions to obtain component I₁;
adding a color-changing material to the component I₁ and mixing uniformly to obtain component I₂; and
adding aerogel to I₂ and mixing uniformly to obtain component I₃.

10. An electrochromic device, comprising the electrochromic material according to claim 1.
